(19) United States
(12) Patent Application Publication
(10) Pub. No.: US 2009/0023771 A1
(43) Pub. Date: Jan. 22, 2009

(54) PHARMACEUTICAL COMPOSITION COMPRISING A PROTON PUMP INHIBITOR AND PROTEIN COMPONENT

(75) Inventor: Jeffrey O. Phillips, Ashland, MO (US)

Correspondence Address: MAYER BROWN LLP, P.O. BOX 2828, CHICAGO, IL, 60690, US

(73) Assignee: The Curators of the University of Missouri, Columbia, MO (US)

(21) Appl. No.: 12/175,266

(22) Filed: Jul. 17, 2008

Related U.S. Application Data
(60) Provisional application No. 60/950,549, filed on Jul. 18, 2007.

(51) Int. Cl. A61K 31/4439; A61K 31/437; A61P 1/04

(52) U.S. Cl. 514/303; 514/338

(57) ABSTRACT

The present disclosure relates to, inter alia, pharmaceutical compositions comprising a H⁺,K⁺-ATPase proton pump inhibitor and a protein component; to methods for manufacture of such compositions, and to use of such compositions in treating and preventing diseases and/or disorders.

### FIELD OF THE INVENTION

The present invention relates to, inter alia, pharmaceutical compositions comprising a H⁺,K⁺-ATPase proton pump inhibitor and a protein component; to methods for manufacture of such compositions, and to use of such compositions in treating and preventing diseases and disorders.

### BACKGROUND

Gastrointestinal disorders such as active duodenal ulcers, gastric ulcers, gastroesophageal reflux disease (GERD), severe erosive esophagitis, poorly responsive symptomatic GERD, nocturnal acid breakthrough (NAB), and pathological hypersecretory conditions such as Zollinger Ellison syndrome represent a major health concern impacting millions of people globally. In fact, it is estimated that as many as 60 million Americans alone experience acid reflux at least once a month, while approximately 19 million Americans suffer from GERD.

In the past, the above-described (and other related) gastrointestinal disorders and their associated symptoms have been treated with H₂ histamine antagonists and antacids. Unfortunately, many such available treatments are not very effective in ameliorating the disorders themselves or their symptoms; additionally, many produce adverse side effects including, among others, constipation, diarrhea and thrombocytopenia. Moreover, H₂ antagonists such as ranitidine and cimetidine are relatively costly modes of therapy.

More recently, at least some of the above-described gastrointestinal disorders have been treated with proton pump inhibitors (also called PPIs). PPIs are believed to reduce gastric acid production by inhibiting H⁺,K⁺-ATPase of the parietal cell—the final common pathway for gastric acid secretion. One particular class of PPIs includes substituted benzimidazole compounds that contain a sulfinyl group bridging substituted benzimidazole and pyridine rings. Another class of PPIs includes imidazopyridine compounds.

At neutral pH, these PPIs are chemically stable, lipid-soluble compounds that have little or no inhibitory activity. It is believed that the neutral PPIs reach parietal cells from the blood and diffuse into the secretory canaliculi where they become protonated and thereby trapped. The protonated agent is then believed to rearrange to form a sulfenic acid and a sulfenamide. The sulfenamide, in turn, is thought to interact covalently with sulfhydryl groups at critical sites in the extracellular (luminal) domain of the membrane-spanning H⁺,K⁺-ATPase. See, Hardman et al., Goodman & Gilman's The Pharmacological Basis of Therapeutics, p. 907, 9th ed. (1996).

Unfortunately, most commercially available proton pump inhibiting compounds are unstable at neutral or acidic pH and undergo decomposition in gastrointestinal fluid upon oral administration, thereby resulting in loss of therapeutic activity. To overcome this acid instability, such compounds are typically formulated for oral delivery as enteric coated solid dosage forms, for example enteric coated tablets, in which coating protects the drug from contact with acidic stomach secretions. An undesirable consequence of such enteric coating is that therapeutic onset time is significantly delayed by comparison with non-enteric coated dosage forms. Such prolonged time to therapeutic onset is particularly undesirable for patients in need of rapid relief from one or more of the above described disorders or symptoms.

For example, U.S. Pat. No. 4,786,505 to Lovgren et al. discloses that a pharmaceutical oral solid dosage form of omeprazole must be protected from contact with acidic gastric juice by an enteric coating to maintain its pharmaceutical activity. That patent describes an enteric coated omeprazole preparation containing an alkaline core comprising omeprazole, a subcoating over the core, and an enteric coating over the subcoating.

More recently, a product containing non-enteric coated PPI has become available in the United States. Zegerid® contains, inter alia, 20 or 40 mg of omeprazole powder and 1680 mgs of sodium bicarbonate. It would be desirable to have additional formulations of PPI that overcome at least some of the above described drawbacks associated with enteric coated dosage forms.

### SUMMARY

In one embodiment, the present disclosure provides a pharmaceutical composition comprising at least one acid labile, H⁺,K⁺-ATPase proton pump inhibitor and a protein component.

In one embodiment, the present disclosure provides a pharmaceutical composition comprising at least one acid labile, H⁺,K⁺-ATPase proton pump inhibitor, at least one buffering agent and a protein component.

In another embodiment, the present disclosure provides a pharmaceutical composition comprising at least one acid labile, H⁺,K⁺-ATPase proton pump inhibitor and L-carnosine.

In yet another embodiment, the present disclosure provides a pharmaceutical composition comprising at least one acid labile, H⁺,K⁺-ATPase proton pump inhibitor, at least one buffering agent and L-carnosine.

In yet another embodiment, the present disclosure provides a pharmaceutical composition comprising at least one acid labile, H⁺,K⁺-ATPase proton pump inhibitor, sodium bicarbonate and L-carnosine.

Also disclosed herein are methods of treating acid related gastrointestinal disorders by administering to a subject one or more compositions described herein.

### BRIEF DESCRIPTION OF THE DRAWINGS

FIG. 1 is a line graph illustrating the affect on pH of 800 mg of L-carnosine, 800 mg sodium bicarbonate and 40 mg omeprazole (20:1 ratio of L-carnosine to omeprazole and sodium bicarbonate to omeprazole).

FIG. 2 is a line graph illustrating the degradation of 40 mg omeprazole when it is administered with 800 mg of L-carnosine and 800 mg sodium bicarbonate (20:1 ratio of L-carnosine to omeprazole and sodium bicarbonate to omeprazole).

FIG. 3 is a line graph illustrating the effect on pH of 1600 mg sodium bicarbonate and 40 mg omeprazole (40:1 ratio).

FIG. 4 is a line graph illustrating the degradation of 40 mg omeprazole when it is administered with 1600 mg sodium bicarbonate (40:1 ratio).

FIG. 5 is a line graph illustrating the affect on pH of 800 mg of aluminum glycinate, 800 mg sodium bicarbonate and 40 mg omeprazole (20:1 ratio of aluminum glycinate to omeprazole and sodium bicarbonate to omeprazole).

FIG. 6 is a line graph illustrating the degradation of 40 mg omeprazole when it is administered with 800 mg of aluminum glycinate and 800 mg sodium bicarbonate (20:1 ratio of aluminum glycinate to omeprazole and sodium bicarbonate to omeprazole).

### DETAILED DESCRIPTION

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any way. Embodiments illustrated under any heading may be combined with embodiments illustrated under any other heading.

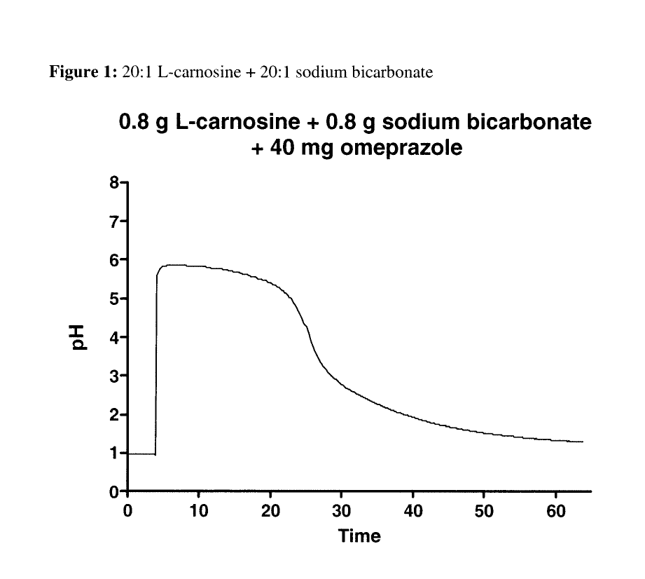

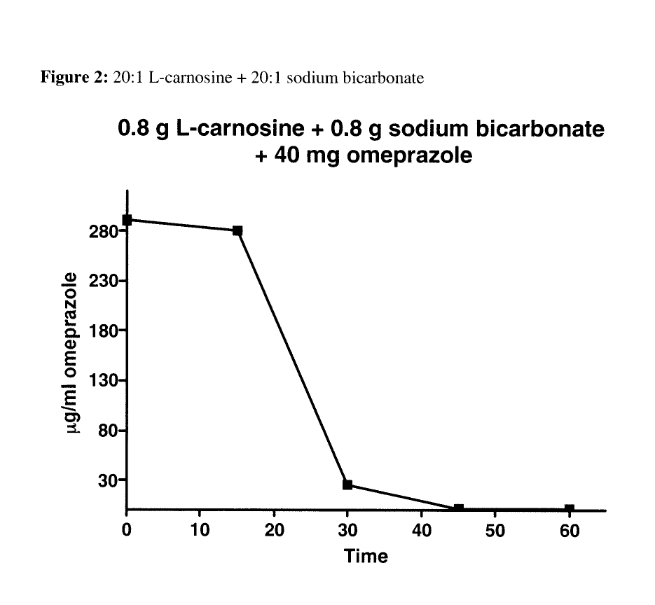

It has been surprisingly found that a pharmaceutical composition comprising at least one acid labile, H⁺,K⁺-ATPase proton pump inhibitor (“PPI”), at least one buffering agent and a protein component prevents immediate degradation of the PPI by stomach acid. See FIGS. 1 and 2.

It has also been surprisingly found that a pharmaceutical composition comprising at least one acid labile, H⁺,K⁺-ATPase proton pump inhibitor (“PPI”), L-carnosine and sodium bicarbonate prevents immediate degradation of the PPI by stomach acid. See FIGS. 1 and 2.

It has further been surprisingly found that a pharmaceutical composition comprising at least one PPI, sodium bicarbonate and L-carnosine, wherein the ratio of sodium bicarbonate to PPI and the ratio of L-carnosine to PPI is about 20 to about 1, prevents immediate degradation of the PPI. See FIGS. 1 and 2.

### Proton Pump Inhibitors

Compositions of the disclosure comprise at least one pharmaceutically acceptable acid labile, H⁺,K⁺-ATPase proton pump inhibitor (“PPI”). The term proton pump inhibitor or PPI means any acid labile pharmaceutical agent possessing pharmacological activity as an inhibitor of H⁺,K⁺-ATPase. A PPI may, if desired, be in the form of free base, free acid, salt, ester, hydrate, anhydrate, amide, enantiomer, isomer, tautomer, prodrug, polymorph, derivative, or the like, provided that the free base, salt, ester, hydrate, amide, enantiomer, isomer, tautomer, prodrug, or any other pharmacologically suitable derivative is therapeutically active or undergoes conversion within or outside of the body to a therapeutically active form.

In one embodiment, illustrative PPIs are those compounds of Formula (I):

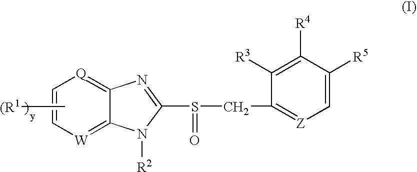

wherein

R¹ is hydrogen, alkyl, halogen, cyano, carboxy, carboalkoxy, carboalkoxyalkyl, carbamoyl, carbamoylalkyl, hydroxy, alkoxy which is optionally fluorinated, hydroxyalkyl, trifluoromethyl, acyl, carbamoyloxy, nitro, acyloxy, aryl, aryloxy, alkylthio, or alkylsulfinyl;

R² is hydrogen, alkyl, acyl, acyloxy, alkoxy, amino, aralkyl, carboalkoxy, carbamoyl, alkylcarbamoyl, dialkylcarbamoyl, alkylcarbonylmethyl, alkoxycarbonylmethyl, or alkylsulfonyl;

R³ and R⁵ are the same or different and each is hydrogen, alkyl, C₁₋₄ lower alkyl (e.g. methyl, ethyl, etc.), alkoxy, amino, or alkoxyalkoxy;

R⁴ is hydrogen, alkyl, C₁₋₄ lower alkyl (e.g. methyl, ethyl, etc.), alkoxy which may optionally be fluorinated, or alkoxyalkoxy;

Q is nitrogen, CH, or CR¹;

W is nitrogen, CH, or CR¹;

y is an integer of 0 through 4; and

Z is nitrogen, CH, or CR

or a free base, salt, ester, hydrate, amide, enantiomer, isomer, tautomer, prodrug, polymorph, or derivative thereof.

Specific examples of suitable PPIs include esomeprazole (also referred to as S-omeprazole), ilaprazole (U.S. Pat. No. 5,703,097), lansoprazole, omeprazole, pantoprazole, pariprazole, rabeprazole, tenatoprazole, leminoprazole and nepaprazole or a free base, a free acid, or a salt, hydrate, ester, amide, enantiomer, isomer, tautomer, polymorph, prodrug, or derivative of such compounds.

Other proton pump inhibitors include but are not limited to: soraprazan (Altana); AZD-0865 (AstraZeneca); YH-1885 (PCT Publication WO 96/05177) (SB-641257) (2-pyrimidinamine, 4-(3,4-dihydro-1-methyl-2(1 H)-isoquinolinyl)-N-(4-fluo-rophenyl)-5,6-dimethyl-monohydrochloride) (YuHan); BY-112 (Altana); SPI-447 (Imidazo(1,2-a)thieno(3,2-c)pyridin-3-amine,5-methyl-2-(2-methyl-3-thieny-1) (Shinnippon); 3-hydroxymethyl-2methyl-9-phenyl-7H-8,9-dihydro-pyrano(2,-3-c)-imidazo(1,2-a)pyridine (PCT Publication WO 95/27714) (AstraZeneca); Pharmaprojects No. 4950 (3-hydroxymethyl-2-methyl-9-phenyl-7H-8,9-dihydro- -pyrano(2,3-c)-imidazo(1,2-a)pyridine) (AstraZeneca, ceased) WO 95/27714; Pharmaprojects No. 4891 (EP 700899) (Aventis); Pharmaprojects No. 4697 (PCT Publication WO 95/32959) (AstraZeneca); H-335/25 (AstraZeneca); T-330 (Saitama 335) (Pharmacological Research Lab); Pharmaprojects No. 3177 (Roche); BY-574 (Altana); Pharmaprojects No. 2870 (Pfizer); AU-1421 (EP 264883) (Merck); AU-2064 (Merck); AY-28200 (Wyeth); Pharmaprojects No. 2126 (Aventis); WY-26769 (Wyeth); pumaprazole (PCT Publication WO 96/05199) (Altana); YH-1238 (YuHan); Pharmaprojects No. 5648 (PCT Publication WO 97/32854) (Dainippon); BY-686 (Altana); YM-020 (Yamanouchi); GYKI-34655 (Ivax); FPL-65372 (Aventis); Pharmaprojects No. 3264 (EP 509974) (AstraZeneca); nepaprazole (To a Eiyo); HN-11203 (Nycomed Pharma); OPC-22575; pumilacidin A (BMS); saviprazole (EP 234485) (Aventis); SKand F-95601 (GSK, discontinued); Pharmaprojects No. 2522 (EP 204215) (Pfizer); S-3337 (Aventis); RS-13232A (Roche); AU-1363 (Merck); SKand F-96067 (EP 259174) (Altana); SUN 8176 (Daiichi Phama); Ro-18-5362 (Roche); ufiprazole (EP 74341) (AstraZeneca); and Bay-p-1455 (Bayer); or a free base, free acid, salt, hydrate, ester, amide, enantiomer, isomer, tautomer, polymorph, prodrug, or derivative of these compounds.

Still other embodiments contemplated by the present disclosure include, but are not limited to those described in the following U.S. Pat. Nos. 4,628,098; 4,689,333; 4,786,505; 4,853,230; 4,965,269; 5,021,433; 5,026,560; 5,045,321; 5,093,132; 5,430,042; 5,433,959; 5,576,025; 5,639,478; 5,703,110; 5,705,517; 5,708,017; 5,731,006; 5,824,339; 5,840,737; 5,855,914; 5,879,708; 5,948,773; 6,017,560; 6,123,962; 6,187,340; 6,296,875; 6,319,904; 6,328,994; 4,255,431; 4,508,905; 4,636,499; 4,738,974; 5,690,960; 5,714,504; 5,753,265; 5,817,338; 6,093,734; 6,013,281; 6,136,344; 6,183,776; 6,328,994; 6,479,075; 6,489,346; 6,559,167; 6,645,988; 6,699,885; 7,101,573; 7,109,161.

Still other embodiments contemplated by the present disclosure include, but are not limited to those described in the following: EP 0254588; EP 0005129.

Other embodiments contemplated by the present disclosure include, but are not limited to those described in the following PCT Publications: WO 94/27988; WO 05/044223; WO 06/043280.

Still other embodiments contemplated by the present disclosure include, but are not limited to those described in the following U.S. Application Nos.: 20020192299; 20040131675; 20040146554; 20040248939; 20040248942; 20050003005; 20050031700; 20050037070; 20050054682; 20050112193; 20050220870; 20050222210; 20050239845; 20050244517; 20050249806; 20050249811; 20050266071; 20050288334; 20050277672; 20050277673; 20050277671; 20060024238; 20060134210; 20060147522; 20060159760; 20060167262; 20060173045; 20060204585.

The foregoing lists of suitable acid inhibitors are meant to be illustrative and not exhaustive as a person of ordinary skill in the art would recognize that there are many other suitable acid inhibitors which could be created.

Gastric acid inhibitors, including proton pump inhibitors as well as their salts, hydrates, esters, amides, enantiomers, isomers, tautomers, polymorphs, prodrugs, and derivatives may be prepared using standard procedures that a person of ordinary skill in the art of synthetic organic chemistry would recognize. See, e.g., March, Advanced Organic Chemistry: Reactions, Mechanisms and Structure, 4th Ed. (New York: Wiley-Interscience, 1992); Leonard et al., Advanced Practical Organic Chemistry (1992); Howarth et al., Core Organic Chemistry (1998); and Weisermel et al., Industrial Organic Chemistry (2002).

“Pharmaceutically acceptable salts,” or “salts,” include the salt of a proton pump inhibitor prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic, methanesulfonic, ethanesulfonic, benzenesulfonic, pantothenic, toluenesulfonic, 2-hydroxyethanesulfonic, sulfanilic, cyclohexylaminosulfonic, algenic, beta.-hydroxybutyric, galactaric and galacturonic acids.

In one embodiment, acid addition salts are prepared from the free base forms using conventional methodology involving reaction of the free base with a suitable acid. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like.

In other embodiments, an acid addition salt is reconverted to the free base by treatment with a suitable base. In a further embodiment, the acid addition salts of the proton pump inhibitors are halide salts, which are prepared using hydrochloric or hydrobromic acids. In still other embodiments, the basic salts are alkali metal salts, e.g., sodium salt.

Salt forms of proton pump inhibitors include, but are not limited to: a sodium salt form such as esomeprazole sodium, omeprazole sodium, rabeprazole sodium, pantoprazole sodium; or a magnesium salt form such as esomeprazole magnesium or omeprazole magnesium, described in U.S. Pat. No. 5,900,424; a calcium salt form; or a potassium salt form such as the potassium salt of esomeprazole, described in U.S. Pat. No. 6,511,996; salt hydrate forms including but not limited to sodium hydrate salt forms, for example tenatoprazole sodium hydrate or omeprazole sodium hydrate. Other salts of esomeprazole are described in U.S. Pat. Nos. 4,738,974 and 6,369,085. Salt forms of pantoprazole and lansoprazole are discussed in U.S. Pat. Nos. 4,758,579 and 4,628,098, respectively.

The foregoing list of suitable salts of proton pump inhibitors is meant to be illustrative and not exhaustive as a person of ordinary skill in the art would recognize that other pharmaceutically acceptable salts of a proton pump inhibitor could be created.

In one embodiment, preparation of esters involves functionalizing hydroxyl and/or carboxyl groups that may be present within the molecular structure of the drug. In another embodiment, the esters are acyl-substituted derivatives of free alcohol groups, e.g., moieties derived from carboxylic acids of the formula RCOOR₁ where, is a lower alkyl group. Esters can be reconverted to the free acids, if desired, by using conventional procedures such as hydrogenolysis or hydrolysis.

“Amides” may be prepared using techniques known to those skilled in the art or described in the pertinent literature. For example, amides may be prepared from esters, using suitable amine reactants, or they may be prepared from an anhydride or an acid chloride by reaction with an amine group such as ammonia or a lower alkyl amine.

“Tautomers” of substituted bicyclic aryl-imidazoles include, e.g., tautomers of omeprazole such as those described in U.S. Pat. Nos. 6,262,085; 6,262,086; 6,268,385; 6,312,723; 6,316,020; 6,326,384; 6,369,087; and 6,444,689.

An exemplary “isomer” of a substituted bicyclic aryl-imidazole is the isomer of omeprazole including but not limited to isomers described in: Oishi et al., Acta Cryst (1989), C45, 1921-1923; U.S. Pat. No. 6,150,380; U.S. Patent Publication No. 02/0156284; and PCT Publication No. WO 02/085889.

Exemplary “polymorphs” include, but are not limited to, those described in PCT Publication No. WO 92/08716, and U.S. Pat. Nos. 4,045,563; 4,182,766; 4,508,905; 4,628,098; 4,636,499; 4,689,333; 4,758,579; 4,783,974; 4,786,505; 4,808,596; 4,853,230; 5,026,560; 5,013,743; 5,035,899; 5,045,321; 5,045,552; 5,093,132; 5,093,342; 5,433,959; 5,464,632; 5,536,735; 5,576,025; 5,599,794; 5,629,305; 5,639,478; 5,690,960; 5,703,110; 5,705,517; 5,714,504; 5,731,006; 5,879,708; 5,900,424; 5,948,773; 5,997,903; 6,017,560; 6,123,962; 6,147,103; 6,150,380; 6,166,213; 6,191,148; 5,187,340; 6,268,385; 6,262,086; 6,262,085; 6,296,875; 6,316,020; 6,328,994; 6,326,384; 6,369,085; 6,369,087; 6,380,234; 6,428,810; 6,444,689; and 6,462,0577.

In one embodiment, at least one proton pump inhibitor is not enteric coated. In another embodiment, a portion of at least one proton pump inhibitor is optionally enteric coated. In one embodiment, no portion of the proton pump inhibitor is enteric coated. In another embodiment, a therapeutically effective portion of at least one proton pump inhibitor is optionally enteric coated. In another embodiment, about 5%, about 15%, about 20%, about 30%, about 40%, about 50% or about 60% of at least one proton pump inhibitor is optionally enteric coated. In another embodiment, a portion of at least one proton pump inhibitor comprises a “thin enteric coat.” The term “thin enteric coat” herein refers to a pH sensitive coating that is applied in a manner or amount such that it delays release of the coated substance in gastrointestinal fluid for a period of time, but ultimately allows release of some of the coated substance prior to passage into the duodenum.

In one embodiment, the proton pump inhibitor has a D₉₀, D₈₀, D₇₀ or D₅₀ particle size, by weight or by number, of less than about 500 μm, less than about 400 μm, less than about 300 μm, less than about 200 μm, less than about 150 μm, less than about 100 μm, less than about 80 μm, less than about 60 μm, less than about 40 μm, less than about 35 μm, less than about 30 μm, less than about 25 μm, less than about 20 μm, less than about 15 μm, or less than about 10 μm.

In another embodiment, compositions are provided wherein the micronized proton pump inhibitor is of a size which allows greater than about 90% or greater than about 75% of the proton pump inhibitor to be released from the dosage unit within about 1 hour, within about 50 minutes, within about 40 minutes, within about 30 minutes, within about 20 minutes, within about 10 minutes, or within about 5 minutes after placement in a standard dissolution test.

In another embodiment, compositions disclosed herein comprise one or more PPIs in a total amount of about 1 mg to about 3000 mg, about 1 mg to about 2000 mg, about 1 mg to about 1000 mg, about 5 mg to about 750 mg, about 5 mg to about 500 mg, about 5 mg to about 250 mg, about 5 mg to about 100 mg, about S mg to about 100 mg, or about 5 mg to about 50 mg, for example about 5 mg, about 7.5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, or about 80 mg.

Compositions of the disclosure can be in the form of an orally deliverable dosage unit. The terms “oral administration” or “orally deliverable” herein include any form of delivery of a therapeutic agent or a composition thereof to a subject wherein the agent or composition is placed in the mouth of the subject, whether or not the agent or composition is swallowed. Thus “oral administration” includes buccal and sublingual as well as esophageal administration.

### Protein Component

Compositions disclosed herein comprise a protein component. The term “protein component” as used herein includes protein isolates, hydrolyzed proteins (protein hydrolysates) as well as protein concentrates. Also included within the definition of a protein component are peptone, tryptone, and peptides. A non-limiting example of a protein is lactoferrin. The term “protein component” does not embrace individual amino acids but can include peptides such as the dipeptide carnosine.

Compositions of the disclosure optionally comprise one or more of a protein isolate, a protein hydrolysate, a protein concentrate, peptone, tryptone, and/or peptides. A suitable protein component can be derived from any origin including plants, animals, or a combination thereof. Non-limiting examples of suitable sources of protein component include soy, corn, whey, egg, casein, fish, meat, poultry etc.

Protein isolate typically comprises at least about 85%, for example about 85-95% protein on a dry basis. Suitable protein isolates can be prepared using any suitable procedure, for example by using an alcohol wash, water wash or ionization concentration techniques that separate at least a portion of carbohydrates and fats from the protein itself.

Protein concentrate typically comprises about 50% to about 85% protein on a dry basis, for example about 60 to about 85%. Protein concentrate can be prepared using any suitable process, for example by concentrating the desired protein through high heat drying (dehydration), acid extraction or filtration to reduce the original source to a more concentrated protein.

Protein hydrolysates are protein molecules that have been lysed, typically but not exclusively with water, into smaller peptides. Protein isolates suitable for the disclosure include substantially pure protein isolate or protein isolate formulations, for example liquid or powder formulations. Non-limiting examples of powder protein hydrolysate formulations include Alimentum, Nutramigen, and Pregestimil.

In one embodiment, compositions of the disclosure comprise a protein component in a total amount of about 1% to about 95%, about 5% to about 90%, or about 10% to about 85%, or about 15% to about 80%, or about 20% to about 75%, or about 25% to about 70%, or about 30% to about 65%, or about 40% to about 60%, on a dry weight basis in the composition, for example, about 1%, about 5%, about 10%, about 15%, about 20%, about 25%, about 30%, about 35%, about 40%, about 45%, about 50%, about 55%, about 60%, about 65%.

In another embodiment, compositions of the disclosure comprise a protein component in a total amount of about 1 mg to about 100 g, about 1 mg to about 20 g, about 1 mg to about 10 g, about 5 mg to about 5 g, about 10 mg to about 2.5 g, about 10 mg to about 1.0 g, or about 10 mg to about 0.5 g on a dry weight basis, for example, about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1 g, about 1.2 g, about 1.3 g, about 1.4 g, about 1.5 g, about 1.6 g, about 1.7 g, about 1.8 g, about 1.9 g, about 2 g, about 2.1 g, about 2.2 g, about 2.3 g, about 2.4 g, about 2.5 g, about 2.6 g, about 2.7 g, about 2.8 g, about 2.9 g, about 3 g, about 3.5 g, about 4 g, about 4.5 g, about 5 g.

In another embodiment, the weight ratio of PPI to protein component, on a dry basis, is about 0.001 to about 1, about 0.0025 to about 0.5, or about 0.1 to about 0.05.

In another embodiment of the disclosure, the protein component has a Protein Digestibility-Corrected Amino Acid Score (PDCAAS) of at least about 0.68, at least about 0.75, at least about 0.80 at least about 0.85, at least about 0.90, at least about 0.92, at least about 0.95, at least about 0.98, or about 1.

In another embodiment of the disclosure, the protein component has a PDCAAS of about 0.68 to about 1, about 0.75 to about 1, about 0.80 to about 1, about 0.90 to about 1, about 0. 92 to about 1 or about 0.95 to about 1.

Without being bound by theory, it is presently believed that upon administration of a composition of the disclosure to a subject, the protein component sacrificially combines with available hydrogen ion (in the GI tract) thereby preventing, slowing or delaying acid-related degradation of the PPI. In another embodiment, therefore, upon administration of a composition of the disclosure to a human subject, the PPI undergoes reduced gastrointestinal degradation by comparison with administration of PPI alone. This can be determined by any suitable method, for example by sampling and assaying contents of the subjects stomach at various time points after ingestion of a composition of the disclosure or a comparative PPI composition comprising no protein component (e.g. naked PPI).

In one embodiment, a composition of the disclosure comprises L-carnosine. Still another embodiment of the disclosure comprises L-carnosine in a ratio of greater than about 20 parts L-carnosine to about 1 part PPI. Other embodiments comprise L-carnosine and PPI in an amount of about 20:1, about 25:1, about 30:1, about 35:1, about 40:1, about 45:1, or about 50:1.

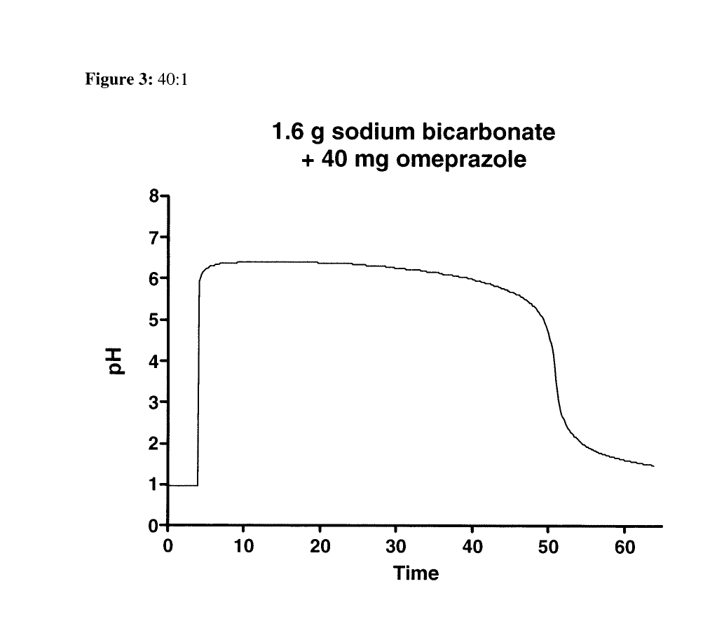

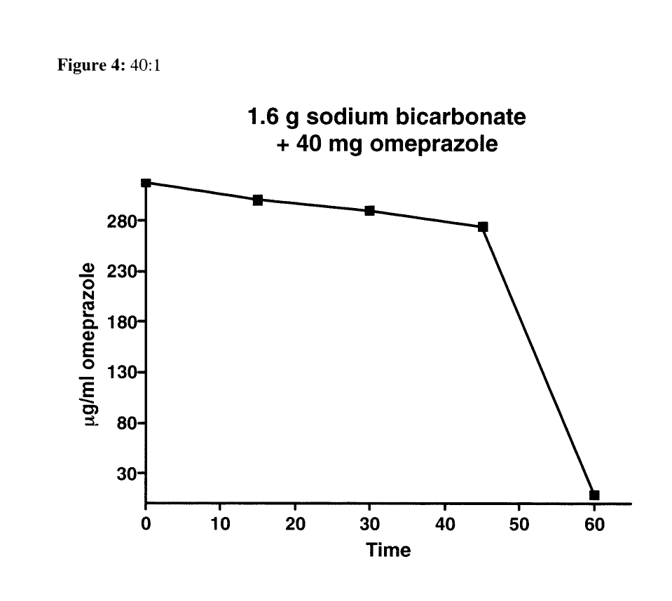

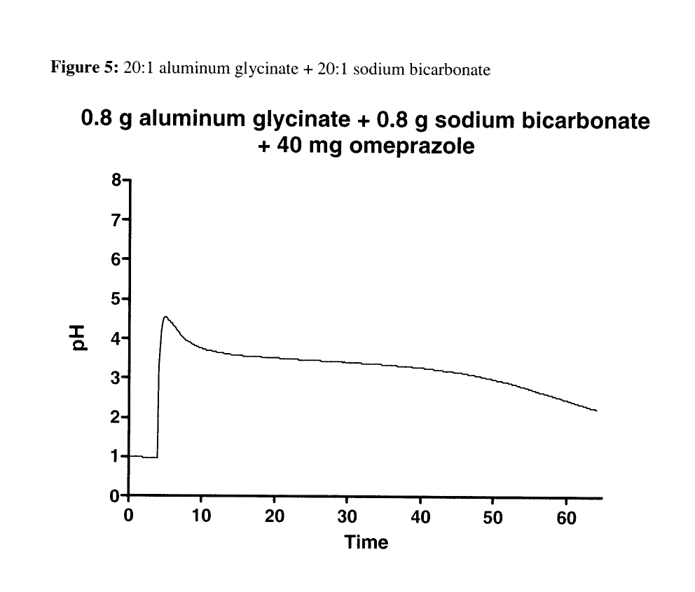

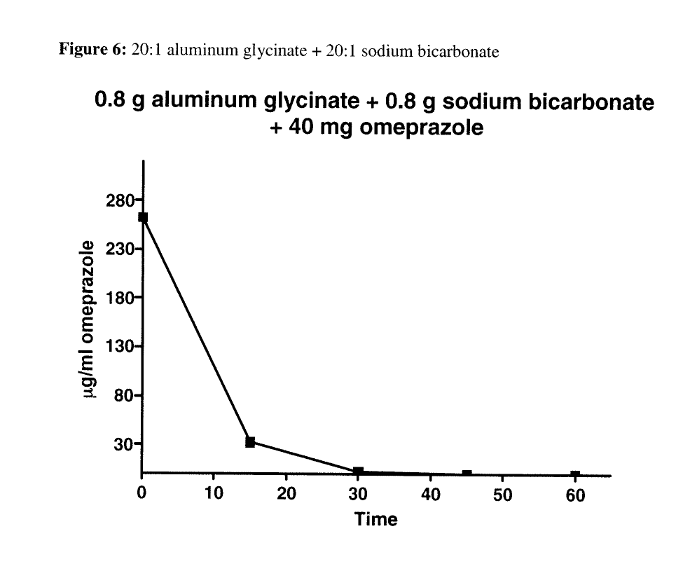

In another embodiment, a composition of the disclosure does not contain an alkali earth metal buffering agent. In still another embodiment, a composition of the disclosure does not contain an alkaline earth metal buffering agent. In another embodiment, a composition of the disclosure does not contain aluminum and/or aluminum glycinate. As illustrated by FIGS. 5 and 6, adding aluminum glycinate to a sodium bicarbonate and omeprazole causes immediate degradation of the omeprazole, as opposed to the degradation of omeprazole when sodium bicarbonate and omeprazole are used alone. See FIGS. 3 and 4.

Other embodiments of the disclosure comprises a PPI, at least one buffering agent in an amount of about 20 parts to about 1 part PPI, and a protein component in an amount of about 20 parts to about 1 part PPI. For example, an embodiment of the disclosure comprises tenatoprazole, sodium bicarbonate in an amount of about 20 parts to about 1 part tenatoprazole, and L-carnosine in an amount of about 20 parts to about 1 part tenatoprazole. Another embodiment of the disclosure comprises about 40 mg tenatoprazole, about 1600 mg sodium bicarbonate, and about 1600 mg L-carnosine. Still another embodiment of the disclosure comprises about 40 mg tenatoprazole, about 1600 mg sodium bicarbonate and magnesium hydroxide, and about 1600 mg L-carnosine. Yet another embodiment of the disclosure comprises about 40 mg omeprazole, about 1600 mg sodium bicarbonate and magnesium hydroxide, and about 1600 mg L-carnosine.

Other embodiments of the disclosure comprise omeprazole, sodium bicarbonate in an amount of about 20 parts to about 1 part omeprazole, and L-carnosine in an amount of about 20 parts to about 1 part omeprazole. For example, an embodiment of the disclosure comprises about 40 mg omeprazole, about 1600 mg sodium bicarbonate, and about 1600 mg L-carnosine.

The foregoing list of suitable protein components is meant to be illustrative and not exhaustive as a person of ordinary skill in the art would recognize that other pharmaceutically acceptable protein components could be created.

### Buffering Agent

Compositions of the disclosure comprise one or more pharmaceutically acceptable buffering agents. Buffering agents useful in the present disclosure include agents possessing pharmacological activity as a weak or strong base. In one embodiment, the buffering agent, when formulated with or administered substantially simultaneously with a PPI, functions to raise the pH of gastrointestinal fluid and thereby to substantially prevent or inhibit acid degradation of the PPI by gastrointestinal fluid for a period of time.

In another embodiment, buffering agents useful in accordance with the present disclosure comprise, but are not limited to, a salt of a Group IA metal including, for example, a bicarbonate salt of a Group IA metal, a carbonate salt of a Group IA metal, an alkaline earth metal buffering agent, an aluminum buffering agent, a calcium buffering agent, a sodium buffering agent, or a magnesium buffering agent. Other suitable buffering agents include alkali (sodium and potassium) or alkaline earth (calcium and magnesium) carbonates, phosphates, bicarbonates, citrates, borates, acetates, phthalates, tartrates, succinates and the like, such as sodium or potassium phosphate, citrate, borate, acetate, bicarbonate and carbonate.

Non-limiting examples of suitable buffering agents include aluminum, magnesium hydroxide, aluminum hydroxide/magnesium hydroxide co-precipitate, calcium acetate, calcium bicarbonate, calcium borate, calcium carbonate, calcium bicarbonate, calcium citrate, calcium gluconate, calcium glycerophosphate, calcium hydroxide, calcium lactate, calcium phthalate, calcium phosphate, calcium succinate, calcium tartrate, dibasic sodium phosphate, dipotassium hydrogen phosphate, dipotassium phosphate, disodium hydrogen phosphate, disodium succinate, dry aluminum hydroxide gel, magnesium acetate, magnesium aluminate, magnesium borate, magnesium bicarbonate, magnesium carbonate, magnesium citrate, magnesium gluconate, magnesium hydroxide, magnesium lactate, magnesium metasilicate aluminate, magnesium oxide, magnesium phthalate, magnesium phosphate, magnesium silicate, magnesium succinate, magnesium tartrate, potassium acetate, potassium carbonate, potassium bicarbonate, potassium borate, potassium citrate, potassium metaphosphate, potassium phthalate, potassium phosphate, potassium polyphosphate, potassium pyrophosphate, potassium succinate, potassium tartrate, sodium acetate, sodium bicarbonate, sodium borate, sodium carbonate, sodium citrate, sodium gluconate, sodium hydrogen phosphate, sodium hydroxide, sodium lactate, sodium phthalate, sodium phosphate, sodium polyphosphate, sodium pyrophosphate, sodium sesquicarbonate, sodium succinate, sodium tartrate, sodium tripolyphosphate, synthetic hydrotalcite, tetrapotassium pyrophosphate, tetrasodium pyrophosphate, tripotassium phosphate, trisodium phosphate, and trometamol. (Based in part upon the list provided in The Merck Index, Merck & Co. Rahway, N.J. (2001)). In addition, due to the ability of the protein component to react with stomach acids, they too can serve as buffering agents in the present embodiments. Furthermore, combinations or mixtures of the above mentioned buffering agents can be used in the pharmaceutical formulations described herein.

Buffering agents also include buffering agents or combinations of buffering agents that interact with HCT (or other acids in the environment of interest) faster than the proton pump inhibitor interacts with the same acids. When placed in a liquid phase such as water, these buffering agents produce and maintain a pH greater than the pKa of the proton pump inhibitor.

The foregoing list of suitable buffering agents is meant to be illustrative and not exhaustive as a person of ordinary skill in the art would recognize that other pharmaceutically acceptable buffering agents could be created.

In various other embodiments, the buffering agent is present in a total amount of about 0.1 mEq/mg to about 5 mEq/mg of the proton pump inhibitor, about 0.5 mEq/mg to about 3 mEq/mg of the proton pump inhibitor, about 0.6 mEq/mg to about 2.5 mEq/mg of the proton pump inhibitor, about 0.7 mEq/mg to about 2.0 mEq/mg of the proton pump inhibitor, about 0.8 mEq/mg to about 1.8 mEq/mg of the proton pump inhibitor, about 1.0 mEq/mg to about 1.5 mEq/mg of the proton pump inhibitor. In another embodiment, the buffering agent is present in an amount of about 0.5 mEq/mg of the proton pump inhibitor, about 0.75 mEq/mg of the proton pump inhibitor, or about 1 mEq/mg of the proton pump inhibitor on a dry weight basis.

In still another embodiment, one or more buffering agents are present in a total amount of about 0.5 mEq to about 160 mEq, about 1 mEq to about 150 mEq, about 10 mEq to about 150 mEq, about 10 mEq to about 75 mEq, about 10 mEq to about 60 mEq, or about 10 mEq to about 50 mEq. Illustratively, a composition of the disclosure can comprise about 1 mEq, about 5 mEq, about 10 mEq, about 15 mEq, about 20 mEq, about 25 mEq, about 30 mEq, about 35 mEq, about 40 mEq, about 45 mEq, about 50 mEq, about 60 mEq, about 70 mEq, about 80 mEq, about 90 mEq, about 100 mEq, about 110 mEq, about 120 mEq, about 130 mEq, about 140 mEq, about 150 mEq, or about 160 mEq of buffering agent.

In yet another embodiment, one or more buffering agents are present in a total amount of about 10 mEq, about 11 mEq, about 12 mEq, about 13 mEq, about 14 mEq, about 15 mEq, or at least about 16 mEq.

In another embodiment, one or more buffering agents and the mixture of the first and subsequent proton pump inhibitors or the salt form of a proton pump inhibitor and the free base form of a proton pump inhibitor are present in a weight ratio of about 5:1, about 7:1, about 10:1, about 20:1, greater than about 20:1, about 21:1, about 22:1, about 23:1, about 25:1, about 30:1, about 35:1, about 40:1, greater than about 40:1, about 45:1, about 53:3; about 11:1, about 28:3, about 28:5, about 23:3, about 26:1, about 27:2, or about 31:1.

In still another embodiment, a first PPI (“PPI1”), a second PPI (“PPI2”), and one or more buffering agents are present in a weight ratio of about 2:1:50, about 3:2:50, about 2:1:25, about 2:1:60, about 3:2:25, about 2:1:20, about 1:1:50, about 1:2:50, about 1:1:25, about 1:1:60, about 1:2:25, or about 1:1:20.

In yet another embodiment, the salt form of a proton pump inhibitor (“PPI-salt”), the free base form of a proton pump inhibitor (“PPI-base”), and one or more buffering agents are present in a weight ratio of about 2:1:50, about 3:2:50, about 2:1:25, about 2:1:60, about 3:2:25, about 2:1:20, about 1:1:50, about 1:2:50, about 1:1:25, about 1:1:60, about 1:2:25, or about 1:1:20.

In another embodiment, the amount of buffering agent present in a composition of the disclosure ranges from about 200 to about 3500 mg, about 300 to about 3000 mg, about 400 to about 2500 mg, or about 500 to about 2200 mg, about 600 to about 2000, or about 700 to about 1800 mg. In other embodiments, the amount of buffering agent present in a composition of the disclosure is about 200 mgs, or about 300 mgs, or about 400 mgs, or about 500 mgs, or about 600 mgs, or about 700 mgs, or about 800 mgs, or about 900 mgs, or about 1000 mgs, or about 1100 mgs, or about 1200 mgs, or about 1300 mgs, or about 1400 mgs, or about 1500 mgs, or about 1600 mgs, or about 1700 mgs, or about 1800 mgs, or about 1900 mgs, or about 2000 mgs, or about 2100 mgs, or about 2200 mgs, or about 2300 mgs, or about 2400 mgs, or about 2500 mgs, or about 2600 mgs, or about 2700 mgs, or about 2800 mgs, or about 2900 mgs, or about 3000 mgs, or about 3200 mgs, or about 3500 mgs.

In another embodiment, one or more buffering agents are present in a composition of the disclosure in a total amount that is greater than 800 mg, for example about 920 mg or at least about 1000 mg.

In still another embodiment, the buffering agent and the mixture of PPI1 and PPI2 or PPI-salt and PPI-base (hereinafter “proton pump inhibitor mixture”) are present in a weight ratio greater than 20:1, not less than about 21:1, not less than about 22:1, not less than about 23:1, not less than about 24:1, not less than about 25:1, not less than about 26:1, not less than about 27:1, not less than about 28:1, not less than about 29:1, not less than about 30:1, not less than about 31:1, not less than about 32:1, not less than about 33:1, not less than about 34:1, not less than about 35:1, not less than about 36:1, not less than about 37:1, not less than about 38:1, not less than about 39:1, not less than about 40:1, not less than about 41:1, not less than about 42:1, not less than about 43:1, not less than about 44:1, not less than about 45:1, not less than about 46:1, not less than about 47:1, not less than about 48:1, not less than about 49:1, not less than about 50:1, not less than about 53:3; not less than about 11:1, not less than about 28:3, not less than about 21:1, not less than about 28:5, not less than about 23:3, not less than about 26:1, not less than about 53:3, not less than about 27:2, or not less than about 31:1.

In yet another embodiment, PPI1, PPI2, and the one or more buffering agents are present in a weight ratio of about 2:1:50, about 3:2:50, about 2:1:25, about 2:1:60, about 3:2:25, about 2:1:20, about 1:1:50, about 1:2:50, about 1:1:25, about 1:1:60, about 1:2:25, or about 1:1:20.

In another embodiment, PPI-salt, PPI-base, and one or more buffering agents are present in a weight ratio of about 2:1:50, about 3:2:50, about 2:1:25, about 2:1:60, about 3:2:25, about 2:1:20, about 1:1:50, about 1:2:50, about 1:1:25, about 1:1:60, about 1:2:25, or about 1:1:20.

In yet another embodiment, a composition is provided that comprises a combination of at least two non-amino acid buffering agents, wherein the combination of at least two non-amino acid buffering agents comprises substantially no aluminum hydroxide-sodium bicarbonate co-precipitate. In a related embodiment, if such a composition comprises a poly[phosphoryl/sulfon]-ated carbohydrate, the weight ratio of poly[phosphory1/sulfon]-ated carbohydrate to buffering agent is less than 1:5 (0.2), less than 1:10 (0.1) or less than 1:20 (0.05). Alternatively, the poly[phosphoryl/sulfon]-ated carbohydrate is present in the composition, if at all, in an amount less than 50 mg, less than 25 mg, less than 10 mg or less than 5 mg. In another embodiment, the composition contains no poly[phosphoryl/sulfon]-ated carbohydrate.

In another embodiment, a composition of the disclosure comprises at least one non-amino acid buffering agent wherein the non-amino acid buffering agent is present in the composition in a total amount greater than 800 mg. In a related embodiment, if such a composition comprises a poly[phosphoryl/sulfon]-ated carbohydrate, the weight ratio of poly[phosphoryl/sulfon]-ated carbohydrate to buffering agent is less than 1:5 (0.2), less than 1:10 (0.1) or less than 1:20 (0.05). Alternatively, the poly[phosphoryl/sulfon]-ated carbohydrate is present in the composition, if at all, in an amount less than 50 mg, less than 25 mg, less than 10 mg or less than 5 mg.

In other embodiments, where two or more buffering agents are present, the two or more buffering agents comprise at least two non-amino acid buffering agents, wherein the combination of at least two non-amino acid buffering agents comprises substantially no aluminum hydroxide-sodium bicarbonate co-precipitate.

In still another embodiment, the buffering agent comprises a mixture of sodium bicarbonate, calcium carbonate, and magnesium hydroxide, wherein the sodium bicarbonate, calcium carbonate, and magnesium hydroxide are each present in an amount of about 0.1 mEq/mg proton pump inhibitor mixture to about 5 mEq/mg of the proton pump inhibitor mixture.

In another embodiment, the buffering agent comprises a mixture of sodium bicarbonate, calcium carbonate, and magnesium hydroxide, wherein the sodium bicarbonate, calcium carbonate, and magnesium hydroxide are each present in an amount of about 0.1 mEq/mg proton pump inhibitor to about 5 mEq/mg of the either proton pump inhibitor.

Also provided herein are pharmaceutical compositions comprising at least one soluble buffering agent. The term “soluble buffering agent” as used herein refers to an antacid that has a solubility of at least about 500 mg/mL, or at least about 300 mg/mL, or at least about 200 mg/mL, or at least about 100 mg/mL in gastrointestinal fluid or simulated gastrointestinal fluid.

In some embodiments, the buffering agent has a defined particle size distribution. For example, in one embodiment, the D₅₀, D₇₀, D8₀, or D₉₀ particle size of the buffering agent, by weight or by number, is no greater than about 10 μm, is no greater than about 20 μm, no greater than about 30 μm, no greater than about 40 μm, no greater than about 50 μm, no greater than about 60 μm, no greater than about 70 μm, no greater than about 80 μm, no greater than about 90 μm, no greater than about 100 μm in diameter, no greater than about 200 μm in diameter, no greater than about 300 μm in diameter, no greater than about 400 μm in diameter, no greater than about 1000 μm in diameter, no greater than about 2000 μm in diameter, no greater than about 3000 μm in diameter, no greater than about 4000 μm in diameter, no greater than about 6000 μm in diameter, or no greater than about 9000 μm in diameter.

The foregoing list of suitable buffering agents is meant to be illustrative and not exhaustive as a person of ordinary skill in the art would recognize that other pharmaceutically acceptable buffering agents could be created.

### Pharmaceutical Excipients

Various embodiments can, if desired, include one or more pharmaceutically acceptable excipients. The term “excipient” herein means any substance, not itself a therapeutic agent, used as a carrier or vehicle for delivery of a therapeutic agent to a subject or added to a pharmaceutical composition to improve its handling or storage properties or to permit or facilitate formation of a dose unit of the composition. Excipients include, by way of illustration and not limitation, diluents, disintegrants, binding agents, adhesives, wetting agents, lubricants, glidants, surface modifying agents, substances added to mask or counteract a disagreeable taste or odor, flavors, dyes, fragrances, and substances added to improve appearance of the composition. Any such excipients can be used in any dosage forms of according to the present disclosure, including liquid, solid or semi-solid dosage forms.

Excipients optionally employed in compositions disclosed herein can be solids, semi-solids, liquids or combinations thereof. Compositions of the disclosure containing excipients can be prepared by any known technique of pharmacy that comprises admixing an excipient with a drug or therapeutic agent.

In various embodiments, compositions optionally comprise one or more pharmaceutically acceptable diluents as excipients. Suitable diluents illustratively include, either individually or in combination, lactose, including anhydrous lactose and lactose monohydrate; starches, including directly compressible starch and hydrolyzed starches (e.g., Celutab™and Emdex™); mannitol; sorbitol; xylitol; dextrose (e.g., Cerelose™ 2000) and dextrose monohydrate; dibasic calcium phosphate dihydrate; sucrose-based diluents; confectioner's sugar; monobasic calcium sulfate monohydrate; calcium sulfate dihydrate; granular calcium lactate trihydrate; dextrates; inositol; hydrolyzed cereal solids; amylose; celluloses including microcrystalline cellulose, food grade sources of α- and amorphous cellulose (e.g., Rexcel™) and powdered cellulose; calcium carbonate; glycine; bentonite; polyvinylpyrrolidone; and the like. Such diluents, if present, constitute in total about 5% to about 99%, about 10% to about 85%, or about 20% to about 80%, of the total weight of the composition. The diluent or diluents selected may exhibit suitable flow properties and, where tablets are desired, compressibility.

The use of extragranular microcrystalline cellulose (that is, microcrystalline cellulose added to a wet granulated composition after a drying step) can be used to improve hardness (for tablets) and/or disintegration time.

In various embodiments, compositions optionally comprise one or more pharmaceutically acceptable disintegrants as excipients, particularly for tablet formulations. Suitable disintegrants include, either individually or in combination, starches, including sodium starch glycolate (e.g., Explotab™ of PenWest) and pregelatinized corn starches (e.g., National™ 1551, National™ 1550, and Colocorn™ 1500), clays (e.g., Veegum™ HV), celluloses such as purified cellulose, microcrystalline cellulose, methylcellulose, carboxymethylcellulose and sodium carboxymethylcellulose, croscarmellose sodium (e.g., Ac-Di-Sol™ of FMC), alginates, crospovidone, and gums such as agar, guar, locust bean, karaya, pectin and tragacanth gums.

Disintegrants may be added at any suitable step during the preparation of the composition, particularly prior to a granulation step or during a lubrication step prior to compression. Such disintegrants, if present, constitute in total about 0.2% to about 30%, about 0.2% to about 10%, or about 0.2% to about 5%, of the total weight of the composition.

Croscarmellose sodium is one example of a disintegrant for tablet or capsule disintegration, and, if present, typically constitutes about 0.2% to about 10%, about 0.2% to about 7%, or about 0.2% to about 5%, of the total weight of the composition.

Various embodiments described herein optionally comprise one or more pharmaceutically acceptable binding agents or adhesives as excipients, particularly for tablet formulations. Such binding agents and adhesives may impart sufficient cohesion to the powder being tableted to allow for normal processing operations such as sizing, lubrication, compression and packaging, but still allow the tablet to disintegrate and the composition to be absorbed upon ingestion. Suitable binding agents and adhesives include, either individually or in combination, acacia; tragacanth; sucrose; gelatin; glucose; starches such as, but not limited to, pregelatinized starches (e.g., National™ 1511 and National™ 1500); celluloses such as, but not limited to, methylcellulose and carmellose sodium (e.g., Tylose™); alginic acid and salts of alginic acid; magnesium aluminum silicate; PEG; guar gum; polysaccharide acids; bentonites; povidone, for example povidone K-15, K-30 and K-29/32; polymethacrylates; HPMC; hydroxypropylcellulose (e.g., Klucel™); and ethylcellulose (e.g., Ethocel™). Such binding agents and/or adhesives, if present, constitute in total about 0.5% to about 25%, about 0.75% to about 15%, or about 1% to about 10%, of the total weight of the composition.

Compositions described herein optionally comprise one or more pharmaceutically acceptable wetting agents as excipients. Non-limiting examples of surfactants that can be used as wetting agents in compositions of the disclosure include quaternary ammonium compounds, for example benzalkonium chloride, benzethonium chloride and cetylpyridinium chloride, dioctyl sodium sulfosuccinate, polyoxyethylene alkylphenyl ethers, for example nonoxynol 9, nonoxynol 10, and octoxynol 9, poloxamers (polyoxyethylene and polyoxypropylene block copolymers), polyoxyethylene fatty acid glycerides and oils, for example polyoxyethylene (8) caprylic/capric mono- and diglycerides (e.g., Labrasol™ of Gattefosse), polyoxyethylene (35) castor oil and polyoxyethylene (40) hydrogenated castor oil; polyoxyethylene alkyl ethers, for example polyoxyethylene (20) cetostearyl ether, polyoxyethylene fatty acid esters, for example polyoxyethylene (40) stearate, polyoxyethylene sorbitan esters, for example polysorbate 20 and polysorbate 80 (e.g., Tween™ 80 of ICI), propylene glycol fatty acid esters, for example propylene glycol laurate (e.g., Lauroglycol™ of Gattefossé), sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof. Such wetting agents, if present, constitute in total about 0.25% to about 15%, about 0.4% to about 10%, or about 0.5% to about 5%, of the total weight of the composition.

Compositions described herein optionally comprise one or more pharmaceutically acceptable lubricants (including anti-adherents and/or glidants) as excipients. Suitable lubricants include, either individually or in combination, glyceryl behapate (e.g., Compritol™ 888); stearic acid and salts thereof, including magnesium (magnesium stearate), calcium and sodium stearates; hydrogenated vegetable oils (e.g., Sterotex™); colloidal silica; talc; waxes; boric acid; sodium benzoate; sodium acetate; sodium fumarate; sodium chloride; DL-leucine; PEG (e.g., Carbowax™ 4000 and Carbowax™ 6000); sodium oleate; sodium lauryl sulfate; and magnesium lauryl sulfate. Such lubricants, if present, constitute in total about 0.1% to about 10%, about 0.2% to about 8%, or about 0.25% to about 5%, of the total weight of the composition.

Suitable anti-adherents include talc, cornstarch, DL-leucine, sodium lauryl sulfate and metallic stearates. Talc is a anti-adherent or glidant used, for example, to reduce formulation sticking to equipment surfaces and also to reduce static in the blend. Talc, if present, constitutes about 0.1% to about 10%, about 0.25% to about 5%, or about 0.5% to about 2%, of the total weight of the composition.

Glidants can be used to promote powder flow of a solid formulation. Suitable glidants include colloidal silicon dioxide, starch, talc, tribasic calcium phosphate, powdered cellulose and magnesium trisilicate.

Compositions described herein can comprise one or more anti-foaming agents. Simethicone is an illustrative anti-foaming agent.

Compositions described herein can comprise one or more flavoring agents, sweetening agents, and/or colorants. Flavoring agents useful in the present embodiments include, without limitation, acacia syrup, alitame, anise, apple, aspartame, banana, Bavarian cream, berry, black currant, butter, butter pecan, butterscotch, calcium citrate, camphor, caramel, cherry, cherry cream, chocolate, cinnamon, citrus, citrus punch, citrus cream, cocoa, coffee, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, MagnaSweet®, maltol, mannitol, maple, menthol, mint, mint cream, mixed berry, nut, orange, peanut butter, pear, peppermint, peppermint cream, Prosweet® Powder, raspberry, root beer, rum, saccharin, safrole, sorbitol, spearmint, spearmint cream, strawberry, strawberry cream, stevia, sucralose, sucrose, Swiss cream, tagatose, tangerine, thaumatin, tutti fruitti, vanilla, walnut, watermelon, wild cherry, wintergreen, xylitol, and combinations thereof, for example, anise-menthol, cherry-anise, cinnamon-orange, cherry-cinnamon, chocolate-mint, honey-lemon, lemon-lime, lemon-mint, menthol-eucalyptus, orange-cream, vanilla-mint, etc.

Sweetening agents that can be used in the present embodiments include, by way of example and not limitation, acesulfame potassium (acesulfame K), alitame, aspartame, cyclamate, cylamate, dextrose, isomalt, MagnaSweet®, maltitol, mannitol, neohesperidine DC, neotame, Prosweet® Powder, saccharin, sorbitol, stevia, sucralose, sucrose, tagatose, thaumatin, xylitol, and the like.

The foregoing excipients can have multiple roles. For example, starch can serve as a filler as well as a disintegrant. The classification of excipients listed herein is not to be construed as limiting in any manner.

### Pharmaceutical Dosage Forms

In various embodiments, compositions can be formulated as solid, liquid or semi-solid dosage forms. In one embodiment, such compositions are in the form of discrete dose units or dosage units. The terms “dose unit” and/or “dosage unit” herein refer to a portion of a pharmaceutical composition that contains an amount of a therapeutic agent suitable for a single administration to provide a therapeutic effect. Such dosage units may be administered one to a small plurality (i.e. 1 to about 4) of times per day, or as many times as needed to elicit a therapeutic response. A particular dosage form can be selected to accommodate any desired frequency of administration to achieve a specified daily dose. Typically one dose unit, or a small plurality (i.e. up to about 4) of dose units, provides a sufficient amount of the active drug (e.g. benzimidazole or imidazopyridine moiety) to result in the desired response or effect.

Non-limiting examples of suitable solid dosage forms include tablets (e.g. suspension tablets, bite suspension tablets, rapid dispersion tablets, chewable tablets, effervescent tablets, bilayer tablets, etc), caplets, capsules (e.g. a soft or a hard gelatin capsule), powder (e.g. a packaged powder, a dispensable powder or an effervescent powder), lozenges, sachets, cachets, troches, pellets, granules, microgranules, encapsulated microgranules, powder aerosol formulations, or any other solid dosage form reasonably adapted for oral administration.

In one embodiment, a composition disclosed herein comprises a multi-layer tablet having a core comprising a proton pump inhibitor; the core is substantially or completely surrounded by the protein component. In one embodiment, the protein component layer completely surrounds the core. In another embodiment, the protein component layer partially surrounds the core. In yet another embodiment, the protein component layer is in contact with a portion of or with all of the surface area of the core.

In still another embodiment, there is one or more intermediate layers in between the core and the protein component. The intermediate layers can comprise any pharmaceutically acceptable material, such as inert and non-pH sensitive coating materials such as polymer based coatings.

In another embodiment, a composition of the disclosure comprises a proton pump inhibitor and a protein component mixed together in powder form and optionally filled into a capsule, for example a hard or soft gelatin or HPMC capsule.

Non-limiting examples of suitable liquid dosage forms include solutions, suspension, elixirs, syrups, liquid aerosol formulations, etc. Alternatively, compositions of the disclosure can also be formulated for rectal, topical, or parenteral (e.g. subcutaneous, intramuscular, intravenous and intradermal or infusion) delivery.

In one embodiment, a liquid composition of the disclosure can be prepared comprising water, PPI and a protein component. In another embodiment, a composition of the disclosure can be prepared as two separate liquids that can be mixed together prior to administration to a subject. In this embodiment, the first liquid comprises de-ionized water and PPI. The second liquid comprises a protein component in water. Alternatively, instead of a second liquid comprising the protein component, a dry protein component could be added to the PPI/de-ionized water mixture prior to administration to a subject.

In another embodiment, a single dosage unit comprises a therapeutically effective amount or a therapeutically and/or prophylactically effective amount of PPI. The term “therapeutically effective amount” or “therapeutically and/or prophylactically effective amount” as used herein refers to an amount of compound or agent that is sufficient to elicit the required or desired therapeutic and/or prophylactic response, as the particular treatment context may require.

It will be understood that a therapeutically and/or prophylactically effective amount of a drug for a subject is dependent inter alia on the body weight of the subject. A “subject” herein to which a therapeutic agent or composition thereof can be administered includes a human subject of either sex and of any age, and also includes any nonhuman animal, particularly a domestic or companion animal, illustratively a cat, dog or a horse.

Tablets are an illustrative dosage form for compositions disclosed herein. Tablets can be prepared according to any of the many relevant, well known pharmacy techniques. In one embodiment, tablets or other solid dosage forms can be prepared by processes that employ one or a combination of methods including, without limitation, (1) dry mixing, (2) direct compression, (3) milling, (4) dry or non-aqueous granulation, (5) wet granulation, or (6) fusion.

The individual steps in the wet granulation process of tablet preparation typically include milling and sieving of the ingredients, dry powder mixing, wet massing, granulation and final grinding. Dry granulation involves compressing a powder mixture into a rough tablet or “slug” on a heavy-duty rotary tablet press. The slugs are then broken up into granular particles by a grinding operation, usually by passage through an oscillation granulator. The individual steps include mixing of the powders, compressing (slugging) and grinding (slug reduction or granulation). Typically, no wet binder or moisture is involved in any of the steps.

In another embodiment, solid dosage forms such as tablets can be prepared by mixing a PPI with at least one protein component as described herein above and, if desired, with one or more optional pharmaceutical excipient to form a substantially homogeneous preformulation blend. The preformulation blend can then be subdivided and optionally further processed (e.g. compressed, encapsulated, packaged, dispersed, etc.) into any desired dosage forms.

Compressed tablets can be prepared by compacting a powder or granulation composition of the disclosure. The term “compressed tablet” generally refers to a plain, uncoated tablet suitable for oral ingestion, prepared by a single compression or by pre-compaction tapping followed by a final compression. Tablets of the present disclosure may be coated or otherwise compounded to provide a dosage form affording the advantage of improved handling or storage characteristics. Any such coating may be selected so as to not substantially delay onset of therapeutic effect of a composition of the disclosure upon administration to a subject. The term “suspension tablet” as used herein refers to a compressed tablet that rapidly disintegrates after placement in water.

In one embodiment, compositions of the disclosure are suitable for rapid onset of therapeutic effect, particularly with respect to the PPI component. In another embodiment, upon oral administration of a composition of the disclosure to a subject, at least a therapeutically effective amount of the PPI is available for absorption in the stomach of the subject. As discussed above, most commercially available PPIs require enteric coating to prevent exposure of the PPI to gastrointestinal fluids (and consequent drug degradation) by way of pH dependent coatings. Such coating, in turn, prevents rapid PPI absorption and therapeutic onset of action. Compositions of the present disclosure, by contrast, do not require enteric coating to maintain drug stability in gastrointestinal fluids and thereby provide for rapid absorption and onset of therapeutic effect. In fact, in one embodiment, a composition comprises at least a therapeutically effective amount of PPI that is not enteric coated.

In another embodiment, upon oral administration of a composition of the disclosure to a plurality of fasted adult human subjects, the subjects exhibit an average Tₘₐₓ of active ingredient, (e.g. PPI) within about 30 seconds to about 90 minutes, within about 1 minute to about 80 minutes, within about 5 minutes to about 60 minutes, within about 10 minutes to about 50 minutes, or within about 15 minutes to about 45 minutes.

In still another embodiment, upon administration of a composition of the disclosure to a plurality of fasted adult human subjects, the subjects exhibit an average plasma concentration of the PPI of at least about 0.1 μg/ml, at least about 0.15 μg/ml, at least about 0.2 μg/ml, at least about 0.3 μg/ml, at least about 0.4 μg/ml, at least about 0.5 μg/ml, at least about 0.6 μg/ml, at least about 0.7 μg/ml, at least about 0.8 μg/ml, at least about 0.9 μg/ml, at least about 1 μg/ml, at least about 1.5 μg/ml, or at least about 2.0 μg/ml at any time within about 90 minutes, within about 75 minutes, within about 60 minutes, within about 55 minutes, within about 50 minutes, within about 45 minutes, within about 40 minutes, within about 35 minutes, within about 30 minutes, within about 25 minutes, within about 20 minutes, within about 17 minutes, within about 15 minutes, within about 12 minutes, or within about 10 minutes after administration.

In yet another embodiment, upon administration of a composition of the disclosure to a plurality of fasted adult human subjects, the subjects exhibit a plasma concentration of active ingredient (e.g. PPI) of at least about 0.1 μg/ml, at least about 0.15 μg/ml, at least about 0.2 μg/ml, at least about 0.3 μg/ml, at least about 0.4 μg/ml, at least about 0.5 μg/ml, at least about 0.6 μg/ml, at least about 0.7 μg/ml, at least about 0.8 μg/ml, at least about 0.9 μg/ml, at least about 1.0 μg/ml, at least about 1.5 μg/ml or at least about 2.0 μg/ml, maintained from about 15 minutes to about 60 minutes after administration, from about 15 minutes to about 90 minutes after administration, from about 15 minutes to about 120 minutes after administration, or from about 15 minutes to about 180 minutes after administration.

In another embodiment, upon administration of a composition of the disclosure to a plurality of fasted adult human subjects, the subjects exhibit at least one of: a mean Cₘₐₓ of PPI of about 500 μg/ml to about 2000 μg/ml, about 600 μg/ml to about 1900 μg/ml, or about 700 ng/ml to about 1800 μg/ml; a mean Tₘₐₓ of PPI of about 0.15 to about 2 hours, about 0.25 to about 1.75 hours, or about 0.3 hours to about 1 hour; and/or a mean AUC_{(0-inf)} of PPI of about 1000 to about 3000 μg*hr/ml, about 1500 to about 2700 μg*hr/ml, or about 1700 to about 2500 μg*hr/ml.

In another embodiment, upon administration of a composition of the disclosure to a plurality of fasted adult human subjects, the subjects exhibit: a mean Cₘₐₓ of PPI of about 500 μg/ml to about 2000 μg/ml, about 600 μg/ml to about 1900 μg/ml, or about 700 μg/ml to about 1800 μg/ml; a mean Tₘₐₓ of PPI of about 0.15 to about 2 hours, about 0.25 to about 1.75 hours, or about 0.3 hours to about 1 hour; and a mean AUC_{(0-inf}) of PPI of about 1000 to about 3000 ng*hr/ml, about 1500 to about 2700 ng*hr/ml, or about 1700 to about 2500 ng*hr/ml.

### Storage Stability

In one embodiment, compositions disclosed herein are in the form of a powder for suspension that can be suspended in a liquid vehicle prior to administration to a subject. Liquid compositions comprising an acid labile PPI and a protein component dissolved and/or suspended in a liquid vehicle comprise another embodiment of the disclosure. Generally, a liquid composition of PPI (without a protein component) would exhibit very short period of stability, even when maintained under refrigerated conditions. This is particularly inconvenient in the hospital setting as fresh batches of suspension are continually required.

Suspension compositions of the disclosure comprise at least one PPI, a protein component, a liquid media (e.g. water, de-ionized water, etc) and one or more optional pharmaceutical excipients. Such compositions, upon storage in a closed container maintained at either room temperature, refrigerated (e.g. about 5-10° C.) temperature, or freezing temperature for a period of about 1, 2, 3, 4, 5, 6, 7, 8, 9, 10, 11, or 12 months, exhibit at least about 90%, at least about 92.5%, at least about 95%, or at least about 97.5% of the original PPI present therein.

### Gastrointestinal Disorders

Compositions of the present disclosure are useful for treating and/or preventing gastrointestinal disorders and acid related gastrointestinal disorders. The phrase “acid related gastrointestinal disorder” or “acid related gastrointestinal disease” refers generally to a disorder or disease that occurs due an imbalance between acid and pepsin production on the one hand, so-called aggressive factors, and mucous, bicarbonate, and prostaglandin production on the other hand, so-called defensive factors. In mammals such disorders include, but are not limited to, duodenal ulcer, gastric ulcer, stress erosions and ulceration, stress-related mucosal damage, gastric and duodenal erosions and ulceration, acid dyspepsia, gastroesophageal reflux disease (GERD), nocturnal acid breakthrough (NAB), severe erosive esophagitis, poorly responsive symptomatic gastroesophageal reflux disease, acid reflux, heartburn, nighttime heartburn symptoms, esophageal ulcers and erosions, Barrett's esophagus, precancerous and cancerous lesions of the esophagus induced by acid exposure, acid hypersecretory conditions, gastrointestinal pathological hypersecretory conditions (such as Zollinger Ellison Syndrome), gastrointestinal bleeding, acute upper gastrointestinal bleeding, non-ulcer dyspepsia, heartburn, ulcers induced by NSAIDs, atypical reflux conditions, laryngitis, chronic cough, otitis media, sinusitis, eye pain, globus sensation, esophagitis, erosive esophagitis, esophageal squamous cell reversion, gastrinoma, Helicobacter pylori (H. pylori) infection, irritable bowel syndrome (IBS), inflammatory bowel disease (IBD), ulcerative colitis, pre- and post-operative acid aspiration, Crohn's disease, asthma, laryngitis, sleep apnea, sleep disturbance, psoriasis, intensive care therapy, and diseases related to any of the above-mentioned conditions are also provided.

Where the disorder is heartburn, the heartburn can be meal-related or induced, sleep-related or induced, and/or nighttime-related or induced heartburn. Sleep-related heartburn and/or nighttime-related heartburn can be caused, for example, by breakthrough gastritis between conventional doses of a therapeutic agent, such as while sleeping or in the early morning hours after a night's sleep. Treatment of these conditions is accomplished by administering to a subject a gastrointestinal-disorder-effective amount (or a therapeutically-effective amount) of a pharmaceutical composition according to the present disclosure. A subject may be experiencing one or more of these conditions or disorders.

Compositions of the disclosure can be administered to a subject at any suitable time, for example upon waking, prior to a meal, during the day, or at night time (e.g. before bed).

The term “treat” or “treatment” as used herein refers to any treatment of a disorder or disease associated with a gastrointestinal disorder, and includes, but is not limited to, preventing the disorder or disease from occurring in a subject that may be predisposed to the disorder or disease but has not yet been diagnosed as having the disorder or disease; inhibiting the disorder or disease, for example, arresting the development of the disorder or disease; relieving the disorder or disease, for example, causing regression of the disorder or disease; or relieving the condition caused by the disease or disorder, for example, stopping the symptoms of the disease or disorder.

The term “prevent” or “prevention,” in relation to a gastrointestinal disorder or disease, means preventing the onset of gastrointestinal disorder or disease development if none had occurred, or preventing further gastrointestinal disorder or disease development if the gastrointestinal disorder or disease was already present.

In one embodiment of the disclosure, a composition of the present disclosure can further include one or more parietal cell activators (in addition to the protein component which may also be a parietal cell activator). Parietal cell activators may be used where the benzimidazole or imidazopyridine moiety is a PPI. Parietal cell activators stimulate the parietal cells and enhance the pharmacologic activity of the PPI administered. For the purposes of this application, “parietal cell activator” or “activator” shall mean any compound or mixture of compounds possessing such stimulatory effect including, but not limited to, chocolate, peppermint oil, spearmint oil, coffee, tea and colas (even if decaffeinated), caffeine, theophylline, theobromine, and combinations thereof.

Parietal cell activators, if desired, are typically present in a composition of the disclosure in an amount sufficient to produce the desired stimulatory effect without causing untoward side effects to patients. For example, chocolate, as raw cocoa, is administered in an amount of about 5 mg to 2.5 g per 20 mg dose of omeprazole (or equivalent pharmacologic dose of another proton pump inhibiting agent). The dose of activator administered to a subject, for example, a human, in the context of the present disclosure should be sufficient to result in enhanced effect of a PPI over a desired time frame.

Illustratively, the approximate effective ranges for various parietal cell activators per 20 mg dose of omeprazole (or equivalent dose of other PPI) include, Chocolate (raw cocoa)—5 mg to 2.5 g; Peppermint oil—(powdered form) 1 mg to 1 g; Spearmint oil—(powdered form) 1 mg to 1 g; Coffee—20 ml to 240 ml; Tea—20 ml to 240 ml; Cola—20 ml to 240 ml; Caffeine—0.5 mg to 1.5 g; Theophylline—0.5 mg to 1.5 g; Theobromine—0.5 mg to 1.5g; Phenylalanine—0.5 mg to 1.5 g; and Tryptophan—0.5 mg to 1.5 g.

### EXAMPLES

The following examples illustrate various embodiments of the present disclosure and are not to be construed as limiting the invention in any way.

### Example 1

Formulation 1 as shown in the following table is prepared as described below.

Formulation 1

| Component | Amount | |
|---|---|---|
| Casein hydrolysate | 4000 | mg (10 mg to 20 g range) |
| Sucrose | 2400 | mg |
| Sucralose | 10 | mg |
| Lactoferrin | 100 | mg |
| Neotame | 3 | mg |
| Omeprazole, | 20 | mg |
| Tenatoprazole, | | |
| Esomeprazole, | | |
| Pantoprazole or | | |
| Lanzoprazole | | |
| Total | 6533 | mg |

The formulation can be suitable for administration in a liquid vehicle or infant formula or as a standalone feeding/medication regime. The formulation can be heat stable such that warming to body temperature (as is normal practice for feeding to infants) has no effect on stability of medication or other additives. When added to infant formula, the entire dry contents may be added without prior reconstitution with water. When used as a stand alone feeding/medication regime, a quantity of water sufficient to bring the total volume to 100 mL can be added.

Alternatively, the formulation can be encapsulated, tableted, or otherwise formed into one or more suitable solid finished dosage form(s). For example, the formulation may further comprise a disintegrant such as starch or croscarmellose sodium in an amount of about 1% to about 6%.

### Example 2

Formulation 2 as shown in the following table is prepared as described below.

Formulation 2

| Component | Amount | |
|---|---|---|
| Casein hydrolysate | 2000 | mg (10 mg to 20 g range) |
| Demineralized whey | 2000 | mg |
| hydrolysate | | |
| Sucrose | 2400 | mg |
| Sucralose | 10 | mg |
| Neotame | 3 | mg |
| Omeprazole, | 10 | mg (1 mg to 4 g range) |
| Tenatoprazole, | | |
| Esomeprazole, | | |
| Pantoprazole or | | |
| Lanzoprazole | | |
| Total | 6423 | mg |

The formulation can be suitable for administration in a liquid vehicle or infant formula or as a standalone feeding/medication regime. The formulation can be heat stable such that warming to body temperature (as is normal practice for feeding to infants) has no effect on stability of medication or other additives. When added to infant formula, the entire dry contents may be added without prior reconstitution with water. When used as a stand alone feeding/medication regime, a quantity of water sufficient to bring the total volume to 100 mL can be added.

Alternatively, the formulation can be encapsulated, tableted, or otherwise formed into one or more suitable solid finished dosage form(s). For example, the formulation may further comprise a disintegrant such as starch or croscarmellose sodium in an amount of about 1% to about 10%.

### Example 3

Formulation 3 as shown in the following table is prepared as described below.

Formulation 3

| Component | Amount | |
|---|---|---|
| Soy hydrolysate | 4000 | mg (10 mg to 20 g range) |
| Demineralized whey | 2000 | mg |
| hydrolysate | | |
| Sucrose | 2400 | mg |
| Lactoferrin | 100 | mg |
| Neotame | 3 | mg |
| Omeprazole, | 10 | mg (1 mg to 5 g range) |
| Tenatoprazole, | | |
| Esomeprazole, | | |
| Pantoprazole or | | |
| Lanzoprazole | | |
| Total | 8513 | |

The formulation can be suitable for administration in a liquid vehicle or infant formula or as a standalone feeding/medication regime. The formulation can be heat stable such that warming to body temperature (as is normal practice for feeding to infants) has no effect on stability of medication or other additives. When added to infant formula, the entire dry contents may be added without prior reconstitution with water. When used as a stand alone feeding/medication regime, a quantity of water sufficient to bring the total volume to 100 mL can be added.

Alternatively, the formulation can be encapsulated, tableted, or otherwise formed into one or more suitable solid finished dosage form(s). For example, the formulation may further comprise a disintegrant such as starch or croscarmellose sodium in an amount of about 1% to about 8%.

### Example 4

Formulations 4-10 as shown in the following table are prepared as described below.

Formulations 4-10

| Component | F4 | | F5 | | F6 | | F7 | | F8 | | F9 | | F10 | |
|---|---|---|---|---|---|---|---|---|---|---|---|---|---|---|
| L-carnosine | 4000 | mg | 4000 | mg | 4000 | mg | 4000 | mg | 4000 | mg | 4000 | mg | 4000 | mg |
| Demineralized | 2000 | mg | 2000 | mg | 2000 | mg | 2000 | mg | 2000 | mg | 2000 | mg | 2000 | mg |
| whey | | | | | | | | | | | | | | |
| hydrolysate | | | | | | | | | | | | | | |
| Sucrose | 2400 | mg | 2400 | mg | 2400 | mg | 2400 | mg | 2400 | mg | 2400 | mg | 2400 | mg |
| Lactoferrin | 100 | mg | 100 | mg | 100 | mg | 100 | mg | 100 | mg | 100 | mg | 100 | mg |
| Neotame | 3 | mg | 3 | mg | 3 | mg | 3 | mg | 3 | mg | 3 | mg | 3 | mg |
| Omeprazole, | 10 | mg | 20 | mg | 40 | mg | 60 | mg | 80 | mg | 90 | mg | 100 | mg |
| Tenatoprazole, | | | | | | | | | | | | | | |
| Esomeprazole, | | | | | | | | | | | | | | |
| Pantoprazole or | | | | | | | | | | | | | | |
| Lanzoprazole | | | | | | | | | | | | | | |
| Total | 8513 | mg | 8523 | mg | 8543 | mg | 8563 | mg | 8583 | mg | 8593 | mg | 8603 | mg |

The formulation can be suitable for administration in a liquid vehicle or infant formula or as a standalone feeding/medication regime. The formulation can be heat stable such that warming to body temperature (as is normal practice for feeding to infants) has no effect on stability of medication or other additives. When added to infant formula, the entire dry contents may be added without prior reconstitution with water. When used as a stand alone feeding/medication regime, a quantity of water sufficient to bring the total volume to 100 mL can be added.

Alternatively, the formulation can be encapsulated, tableted, or otherwise formed into one or more suitable solid finished dosage form(s). For example, the formulation may further comprise a disintegrant such as starch or croscarmellose sodium in an amount of about 2% to about 10%.

### Example 5

Formulation 11 as shown in the following table is prepared as described below.

Formulation 11

| Component | Amount | |
|---|---|---|
| Whey hydrolysate | 4000 | mg (10 mg to 20 g range) |
| Hydrolyzed guar gum | 1000 | mg(1 mg to 20 g range) |
| Sucrose | 2000 | mg |
| Thaumatin | 3 | mg |
| Neotame | 2 | mg |
| Omeprazole, | 10 | mg (1 mg to 5 g range) |
| Tenatoprazole, | | |
| Esomeprazole, | | |
| Pantoprazole or | | |
| Lanzoprazole | | |
| Total | 7015 | mg |

The formulation can be suitable for administration in a liquid vehicle or infant formula or as a standalone feeding/medication regime. The formulation can be heat stable such that warming to body temperature (as is normal practice for feeding to infants) has no effect on stability of medication or other additives. When added to infant formula, the entire dry contents may be added without prior reconstitution with water. When used as a stand alone feeding/medication regime, a quantity of water sufficient to bring the total volume to 100 mL can be added.

Alternatively, the formulation can be encapsulated, tableted, or otherwise formed into one or more suitable solid finished dosage form(s). For example, the formulation may further comprise a disintegrant such as starch or croscarmellose sodium in an amount of about 2% to about 10%.

### Example 6

Formulations 5-11 as shown in the following table are prepared as described below.

Formulations 5-11

| Component | F5 | | F6 | | F7 | | F8 | | F9 | | F10 | | F11 | |
|---|---|---|---|---|---|---|---|---|---|---|---|---|---|---|
| L-carnosine | 4000 | mg | 4000 | mg | 4000 | mg | 4000 | mg | 4000 | mg | 4000 | mg | 4000 | mg |
| Hydrolyzed | 1000 | mg | 1000 | mg | 1000 | mg | 1000 | mg | 1000 | mg | 1000 | mg | 1000 | mg |
| guar gum | | | | | | | | | | | | | | |
| Sucrose | 2000 | mg | 2000 | mg | 2000 | mg | 2000 | mg | 2000 | mg | 2000 | mg | 2000 | mg |
| Thaumatin | 3 | mg | 3 | mg | 3 | mg | 3 | mg | 3 | mg | 3 | mg | 3 | mg |
| Neotame | 2 | mg | 2 | mg | 2 | mg | 2 | mg | 2 | mg | 2 | mg | 2 | mg |
| Omeprazole, | 10 | mg | 20 | mg | 40 | mg | 60 | mg | 80 | mg | 90 | mg | 100 | mg |
| Tenatoprazole, | | | | | | | | | | | | | | |
| Esomeprazole, | | | | | | | | | | | | | | |
| Pantoprazole or | | | | | | | | | | | | | | |
| Lanzoprazole | | | | | | | | | | | | | | |
| Total | 7015 | mg | 7025 | mg | 7045 | mg | 7065 | mg | 7085 | mg | 7095 | mg | 7100 | mg |

The formulation can be suitable for administration in a liquid vehicle or infant formula or as a standalone feeding/medication regime. The formulation can be heat stable such that warming to body temperature (as is normal practice for feeding to infants) has no effect on stability of medication or other additives. When added to infant formula, the entire dry contents may be added without prior reconstitution with water. When used as a stand alone feeding/medication regime, a quantity of water sufficient to bring the total volume to 100 mL can be added.

Alternatively, the formulation can be encapsulated, tableted, or otherwise formed into one or more suitable solid finished dosage form(s). For example, the formulation may further comprise a disintegrant such as starch or croscarmellose sodium in an amount of about 1% to about 10%.

### Example 7

Formulation 12 as shown in the following table is prepared as described below.

Formulation 12

| Component | Amount | |
|---|---|---|
| Sodium Caseinate | 3000 | mg (10 mg to 20 g range) |
| Sucralose | 600 | mg |
| Dextrose | 400 | mg |
| Omeprazole, | 30 | mg (1 mg to 5 g range) |
| Tenatoprazole, | | |
| Esomeprazole, | | |
| Pantoprazole or | | |
| Lanzoprazole | | |
| Total | 4030 | mg |

The formulation can be packaged as a dosage packet for addition to coffee or other beverage or food where a creamer is desired. The formulation may also be formed into a square (such as a typical “sugar cube”) or into a rapidly dissolvable tablet or square (with the addition of hydroxypropyl cellulose, hypromellose or other disintegrants as known by those skilled in the art). Alternatively, the formulation can be encapsulated, tableted, or otherwise formed into one or more suitable solid finished dosage form(s). For example, the formulation may further comprise a disintegrant such as starch or croscarmellose sodium in an amount of about 1% to about 10%.

### Example 8

Formulation 13 as shown in the following table is prepared as described below.

Formulation 13

| Component | Amount | |
|---|---|---|
| Calcium Caseinate | 3000 | mg (10 mg to 20 g range) |
| Sucralose | 200 | mg |
| Dextrose | 200 | mg |
| Aspartame | 200 | mg |
| Omeprazole, | 20 | mg (1 mg to 5 g range) |
| Tenatoprazole, | | |
| Esomeprazole, | | |
| Pantoprazole or | | |
| Lanzoprazole | | |
| Total | 3620 | mg |

The formulation can be packaged as a dosage packet for addition to coffee or other beverage or food where a creamer is desired. The formulation may also be formed into a square (such as a typical “sugar cube”) or into a rapidly dissolvable tablet or square (with the addition of hydroxypropyl cellulose, hypromellose or other disintegrants as known by those skilled in the art).

Alternatively, the formulation can be encapsulated, tableted, or otherwise formed into one or more suitable solid finished dosage form(s). For example, the formulation may further comprise a disintegrant such as starch or croscarmellose sodium in an amount of about 1% to about 10%.

### Example 9

Formulations 14-20 as shown in the following table are prepared as described below.

Formulations 14-20

| Component | F14 | | F15 | | F16 | | F17 | | F18 | | F19 | | F20 | |
|---|---|---|---|---|---|---|---|---|---|---|---|---|---|---|
| L-carnosine | 3000 | mg | 3000 | mg | 3000 | mg | 3000 | mg | 3000 | mg | 3000 | mg | 2800 | mg |
| Sucralose | 200 | mg | 200 | mg | 200 | mg | 200 | mg | 200 | mg | 200 | mg | 200 | mg |
| Dextrose | 200 | mg | 200 | mg | 200 | mg | 200 | mg | 200 | mg | 200 | mg | 200 | mg |
| Aspartame | 200 | mg | 200 | mg | 200 | mg | 200 | mg | 200 | mg | 200 | mg | 200 | mg |
| Omeprazole, | 10 | mg | 20 | mg | 40 | mg | 60 | mg | 80 | mg | 90 | mg | 100 | mg |
| Tenatoprazole, | | | | | | | | | | | | | | |
| Esomeprazole, | | | | | | | | | | | | | | |
| Pantoprazole or | | | | | | | | | | | | | | |
| Lanzoprazole | | | | | | | | | | | | | | |
| Total | 3610 | mg | 3620 | mg | 3640 | mg | 3660 | mg | 3680 | mg | 3690 | mg | 3500 | mg |

The formulation can be packaged as a dosage packet for addition to coffee or other beverage or food where a creamer is desired. The formulation may also be formed into a square (such as a typical “sugar cube”) or into a rapidly dissolvable tablet or square (with the addition of hydroxypropyl cellulose, hypromellose or other disintegrants as known by those skilled in the art).

Alternatively, the formulation can be encapsulated, tableted, or otherwise formed into one or more suitable solid finished dosage form(s). For example, the formulation may further comprise a disintegrant such as starch or croscarmellose sodium in an amount of about 1 to 10%.

### Example 10

Formulation 21 as shown in the following table is prepared as described below.

Formulation 21

| Component | Amount | |
|---|---|---|
| Hydrolyzed Whey isolate | 3000 | mg (10 mg to 20 g range) |
| Sucralose | 200 | mg |
| Dextrose | 200 | mg |
| Aspartame | 200 | mg |
| Neotame | 3 | mg |
| Omeprazole, | 40 | mg (1 mg to 5 g range) |
| Tenatoprazole, | | |
| Esomeprazole, | | |
| Pantoprazole or | | |
| Lanzoprazole | | |
| Total | 3643 | mg |

The formulation can be packaged as a dosage packet for addition to coffee or other beverage or food where a creamer is desired. The formulation may also be formed into a square (such as a typical “sugar cube”) or into a rapidly dissolvable tablet or square (with the addition of hydroxypropyl cellulose, hypromellose or other disintegrants as known by those skilled in the art).

Alternatively, the formulation can be encapsulated, tableted, or otherwise formed into one or more suitable solid finished dosage form(s). For example, the formulation may further comprise a disintegrant such as starch or croscarmellose sodium in an amount of about 1% to about 10%.

### Example 11

Formulations 22-28 as shown in the following table are prepared as described below.

Formulations 22-28

| Component | F22 | | F23 | | F24 | | F25 | | F26 | | F27 | | F28 | |
|---|---|---|---|---|---|---|---|---|---|---|---|---|---|---|
| L-carnosine | 800 | mg | 800 | mg | 800 | mg | 1200 | mg | 1600 | mg | 1800 | mg | 2000 | mg |
| Sucralose | 200 | mg | 200 | mg | 200 | mg | 200 | mg | 200 | mg | 200 | mg | 200 | mg |
| Dextrose | 200 | mg | 200 | mg | 200 | mg | 200 | mg | 200 | mg | 200 | mg | 200 | mg |
| Aspartame | 200 | mg | 200 | mg | 200 | mg | 200 | mg | 200 | mg | 200 | mg | 200 | mg |
| Neotame | 2 | g | 2 | g | 2 | g | 2 | g | 1.5 | g | 1000 | mg | 1000 | mg |
| Omeprazole, | 10 | mg | 20 | mg | 40 | mg | 60 | mg | 80 | mg | 90 | mg | 100 | mg |
| Tenatoprazole, | | | | | | | | | | | | | | |
| Esomeprazole, | | | | | | | | | | | | | | |
| Pantoprazole or | | | | | | | | | | | | | | |
| Lanzoprazole | | | | | | | | | | | | | | |
| Total | 3410 | mg | 3420 | mg | 3440 | mg | 3860 | mg | 3780 | mg | 3490 | mg | 3700 | mg |

The formulation can be packaged as a dosage packet for addition to coffee or other beverage or food where a creamer is desired. The formulation may also be formed into a square (such as a typical “sugar cube”) or into a rapidly dissolvable tablet or square (with the addition of hydroxypropyl cellulose, hypromellose or other disintegrants as known by those skilled in the art).

Alternatively, the formulation can be encapsulated, tableted, or otherwise formed into one or more suitable solid finished dosage form(s). For example, the formulation may further comprise a disintegrant such as starch or croscarmellose sodium in an amount of about 1% to about 10%.

### Example 12

The following Formulations 29-35 are prepared as described below.

Formulations 29-35

| Component | F29 | | F30 | | F31 | | F32 | | F33 | | F34 | | F35 | |
|---|---|---|---|---|---|---|---|---|---|---|---|---|---|---|
| L-carnosine | 800 | mg | 800 | mg | 800 | mg | 1200 | mg | 1600 | mg | 1800 | mg | 2000 | mg |
| Sodium | 800 | mg | 800 | mg | 800 | mg | 1200 | mg | 1600 | mg | 1800 | mg | 2000 | mg |
| Bicarbonate | | | | | | | | | | | | | | |
| Omeprazole, | 10 | mg | 20 | mg | 40 | mg | 60 | mg | 80 | mg | 90 | mg | 100 | mg |
| Tenatoprazole, | | | | | | | | | | | | | | |
| Esomeprazole, | | | | | | | | | | | | | | |
| Pantoprazole or | | | | | | | | | | | | | | |
| Lanzoprazole | | | | | | | | | | | | | | |
| Total | 1610 | mg | 1620 | mg | 1640 | mg | 2460 | mg | 3280 | mg | 3690 | mg | 4100 | mg |

The formulation may be prepared as solid, liquid or semi-solid dosage forms as described hereinabove. For example, the formulation may be tableted or encapsulated or prepared as other dosage forms (with optional excipients). The formulation may further include disintegrants such as crosslinked polyvinylpyrrolidone (Crospovidone USP/NF) in an amount, for example, of about 1% to about 10% weight to weight.

Alternatively, other disintegrants include sodium CMC (carboxymethyl cellulose), chitin, or chitosan. The formulation may further include one or more flavoring agents as described hereinabove, for example, sucralose, dextrose, aspartame, thaumatin or neotame.

All references, including publications, patent applications, and patents, cited herein are hereby incorporated by reference to the same extent as if each reference there individually and specifically indicated to be incorporated by reference and were set forth in its entirety herein.

The use of the terms “a” and “an” and “the” and similar referents in the context of this disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., such as, preferred, preferably, particularly) provided herein, is intended merely to further illustrate the content of the disclosure and does not pose a limitation on the scope of the claims. No language in the specification should be construed as indicating any non-claimed element as essential to the practice of the claimed invention.

Alternative embodiments of the claimed invention are described herein, including the best mode known to the inventors for carrying out the claimed invention. Of these, variations of the disclosed embodiments will become apparent to those of ordinary skill in the art upon reading the foregoing disclosure. The inventors expect skilled artisans to employ such variations as appropriate, and the inventors intend for the claimed invention to be practiced otherwise than as specifically described herein.

Accordingly, the claimed invention includes all modifications and equivalents of the subject matter recited in the claims appended hereto as permitted by applicable law. Moreover, any combination of the above-described elements in all possible variations thereof is encompassed by the claimed invention unless otherwise indicated herein or otherwise clearly contradicted by context.

The use of individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the disclosed subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value. Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Thus, recitation of ranges of values herein are merely intended to serve as a shorthand method of referring individually to each separate value falling within the range, unless otherwise indicated herein, and each separate value is incorporated into the specification as if it there individually recited herein.

It is to be understood that any ranges, ratios and ranges of ratios that can be formed by, or derived from, any of the data disclosed herein represent further embodiments of the present disclosure and are included as part of the disclosure as though they were explicitly set forth. This includes ranges that can be formed that do or do not include a finite upper and/or lower boundary. Accordingly, a person of ordinary skill in the art most closely related to a particular range, ratio or range of ratios will appreciate that such values are unambiguously derivable from the data presented herein.

What is claimed is:

1. A pharmaceutical composition comprising an acid labile proton pump inhibitor and a protein component.

2. The composition of claim 1 wherein the proton pump inhibitor is of Formula (1): 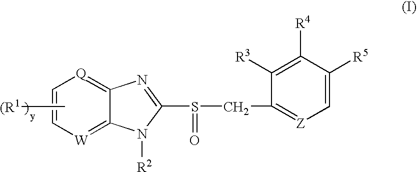
wherein
R¹ is hydrogen, alkyl, halogen, cyano, carboxy, carboalkoxy, carboalkoxyalkyl, carbamoyl, carbamoylalkyl, hydroxy, alkoxy which is optionally fluorinated, hydroxyalkyl, trifluoromethyl, acyl, carbamoyloxy, nitro, acyloxy, aryl, aryloxy, alkylthio, or alkylsulfinyl;
R² is hydrogen, alkyl, acyl, acyloxy, alkoxy, amino, aralkyl, carboalkoxy, carbamoyl, alkylcarbamoyl, dialkylcarbamoyl, alkylcarbonylmethyl, alkoxycarbonylmethyl, or alkylsulfonyl;
R³ and R⁵ are the same or different and each is hydrogen, alkyl, alkoxy, amino, or alkoxyalkoxy;
R⁴ is hydrogen, alkyl, alkoxy which may optionally be fluorinated, or alkoxyalkoxy;
Q is nitrogen, CH, or CR¹;
W is nitrogen, CH, or CR¹;
y is an integer of 0 through 4; and
Z is nitrogen, CH, or CR¹;
or a free base, salt, ester, hydrate, amide, enantiomer, isomer, tautomer, prodrug, polymorph, or derivative thereof.

3. The composition of claim 1 wherein the proton pump inhibitor is omeprazole, tenatoprazole, lansoprazole, rabeprazole, esomeprazole (also referred to as S-omeprazole), pantoprazole, pariprazole, leminoprazole and nepaprazole or a free base, a free acid, or a salt, hydrate, ester, amide, enantiomer, isomer, tautomer, polymorph, prodrug, or derivative of such compounds.

4. The composition of claim 1 wherein the proton pump inhibitor is omeprazole or a free base, a free acid, or a salt, hydrate, ester, amide, enantiomer, isomer, tautomer, polymorph, prodrug, or derivative of such compounds.

5. The composition of claim 1 wherein the proton pump inhibitor is tenatoprazole or a free base, a free acid, or a salt, hydrate, ester, amide, enantiomer, isomer, tautomer, polymorph, prodrug, or derivative of such compounds.

6. The composition of claim 1 wherein the proton pump inhibitor is present in an amount of about 1 mg to about 1000 mg.

7. The composition of claim 1 wherein the proton pump inhibitor is present in an amount of about 15 mg to about 50 mg.

8. The composition of claim 1 wherein the protein component is present in an amount of about 1 mg to about 100 g on a dry weight basis.

9. The composition of claim 1 wherein the protein component is present in an amount of about 10 mg to about 500 mg on a dry weight basis.

10. The composition of claim 1 wherein the proton pump inhibitor and the protein component are present in the composition in a dry weight ratio of about 0.001:1.

11. The composition of claim 1 wherein the proton pump inhibitor and the protein component are present in the composition in a dry weight ratio of about 0.1:0.5.

12. The composition of claim 1 wherein the proton component comprises protein concentrate, protein isolate and/or protein hydrolysate.

13. The composition of claim 1 wherein the protein component is L-carnosine.

14. The composition of claim 1 further comprising at least one pharmaceutically acceptable excipient.

15. The composition of claim 1 wherein the composition comprises a solid dosage forms selected from a tablet, a suspension tablet, a bite suspension tablet, a rapid dispersion tablet, a chewable tablet, an effervescent tablet, a bilayer tablet, a caplet, a capsule, a powder, a lozenge, a sachet, a cachet, a troche, a pellet, a granule and a microgranule.

16. The composition of claim 1 wherein the composition comprises a bi-layer tablet having a core comprising said proton pump inhibitor and a outer layer comprising the protein component, wherein said outer layer substantially completely surrounds the core.

17. A method of treating or preventing an acid related gastrointestinal disorder, the method comprising administering to a subject in need thereof a therapeutically effective amount of a composition of claim 1.

18. A pharmaceutical composition comprising an acid labile proton pump inhibitor and a protein component, wherein: upon administration the composition to a plurality of fasted adult human subjects, the subjects exhibit an average plasma concentration of the PPI of at least about 0.1 μg/ml at any time within about 90 minutes.
